(19) United States
(12) Patent Application Publication
(10) Pub. No.: US 2011/0105441 A1
(43) Pub. Date: May 5, 2011

(54) Stable Orally Disintegrating Tablets Having Low Superdisintegrant

(75) Inventors: Yeli Zhang, Princeton, NJ (US); Brian Carlin, Pittsgrove, NJ (US)

(73) Assignee: FMC CORPORATION, Philadelphia, PA (US)

(21) Appl. No.: 12/702,846

(22) Filed: Feb. 9, 2010

Related U.S. Application Data
(60) Provisional application No. 61/258,866, filed on Nov. 6, 2009.
(60) Provisional application No. 61/280,134, filed on Oct. 30, 2009.

(51) Int. Cl. A61K 31/616; A61P 43/00

(52) U.S. Cl. 514/161

(57) ABSTRACT

The invention is directed to the functionality and performance of superdisintegrants in orally disintegrating tablets (ODT). The invention can be an aged direct compression ODT having between about 0.3% to about 2% (wt/wt) sodium croscarmellose relative to the total weight of the ODT, a polyol matrix, optionally a lubricant, and an active pharmaceutical or nutraceutical ingredient, in which after storage for four months the ODT has a disintegration time using an excess water test that is less than 30 seconds and a tensile strength greater than 0.5 MPa. The invention is also directed to a direct compression ODT, consisting essentially of about 0.5% to 2.0% sodium croscarmellose, from 0.1% to 2.0% lubricant, an API, up to 10% (wt/wt) microcrystalline cellulose, optionally one or more colorants, sweeteners, fragrances, flavor compounds, and/or flavor blockers, and the balance spray-dried mannitol.

### FIELD OF THE DISCLOSURE

The invention is generally directed to an orally disintegrating tablet (ODT) suitable for administration of pharmaceuticals or nutraceuticals. More specifically, the invention is directed to ODTs having relatively low levels of superdisintegrants.

### BACKGROUND

An ODT is a solid dosage form that disintegrates and dissolves in the mouth within 60 seconds or less. Van Arnum, “Advancing ODT technology,” Pharmaceutical Technology, Oct. 2, 2007. ODTs are different from conventional sublingual tablets, lozenges, and buccal tablets which require more than a minute to dissolve in the mouth.

According to US Food and Drug Administration (FDA) guidance, ODTs should have an in vitro disintegration time of 30 seconds or less, based on the United States Pharmacopeia disintegration test method. FDA, Guidance for Industry: Orally Disintegrating Tablets draft guidance, (Rockville, Md., April 2007). At present, ODTs are the only quick-dissolving dosage form recognized by FDA and listed in the Orange Book. Pfister, Ghosh, “Orally disintegrating tablets,” Pharmaceutical Technology, Oct. 2, 2005.

ODTs have advantages as an alternative to conventional oral dosage forms, such as conventional tablets and capsules, particularly convenient administration and increased patient compliance. For example, recent market studies indicate that more than half of the patient population prefers ODTs to conventional tablets or capsules. One very practical reason is that many patients, like children and the elderly, have difficulty swallowing tablets and capsules. There are other patients who simply prefer the convenience of a readily administered dosage form like ODT.

ODTs can be made by various methods, including direct compression, lyophilization, and molding technologies. Dobetti, “Fast-melting tablets: developments and technologies,” Pharm. Technol. 25 (9 Suppl), 44-50 (2001); Sastry, Nyshasham, “Process development and scale-up of oral fast-dissolving tablets,” in Drug Delivery to the Oral Cavity: Molecules to Market, T. K. Ghosh and W. R. Pfister, Eds. (CRC Press, New York, N.Y., 2005), pp. 311-336; Sharma et al., “Manufacturing technology choices for mouth dissolving tablets,” Pharm. Technol. 27 (10 Suppl). 10-13 (2003). Of these different manufacturing processes, direct compression is the most economical method as it uses conventional equipment, commercial available excipients, and relatively simple process steps. Therefore, direct compression is a preferred method.

ODTs have one or more disintegrants to ensure rapid disintegration.

In addition to ODT disintegration time, other parameters are critical for ODT function, including the nature of the matrix, ODT weight, friability, and ODT hardness. Mouth feel is important in consumer or patient acceptance and includes aspects of residual granularity, gumminess, sweetness, bitterness, or medicinal taste, in addition to rapidity of disintegration and dissolution. Some ODTs are made with hard granules such as silica, which generally increase the speed of disintegration but can also lead to consumer avoidance. Generally, the disintegrant has a major role in the disintegration process, and the disintegrant use level will impact ODT hardness and mouth feel. Therefore, the choice of a suitable disintegrant and an optimal use level are critical to ensure a high disintegration rate.

Some disintegrants, also known as superdisintegrants, are particularly effective in inducing rapid ODT disintegration. Superdisintegrants include croscarmellose sodium, for example, Ac-Di-Sol® (FMC Corporation), crospovidone, for example Polyplasdone XL-10® or PVP XL-10, and sodium starch glycolate, for example Glycolys®.

Current ODTs that are made with inorganic fillers suffer from several shortcomings, including relatively poor consumer or patient acceptance. Moreover, ODTs made with low levels of superdisintegrants have had an inadequate combination of properties.

There have been several patents, patent applications, and other publications directed to ODTs.

Ferran, US 20060165781, discloses orally disintegrating tablets and a process for obtaining them.

Martani, U.S. Pat. No. 7,182,959, discloses a rapidly dissolving dosage form and a process for making the same.

Withiam et al., US 20070196476, discloses rapidly dissolving tablets comprising low surface area titanium dioxide.

Ferrari et al. studied the influence of porosity and formula solubility on disintegrant efficiency in tablets. Ferrari, Bertoni, Bonferoni, Rossi, Gassaniga, Conte, and Caramella, S.T.P. Pharma Sciences 5(2):116-121 (1995).

Sandri et al. reviewed differentiating factors between oral fast-dissolving technologies. Sandri, Bonferoni, Ferrari, Rossi, and Carmella, Am. J. Drug Deliv. 4(4):249-262 (2006).

Ohrem et al. discuss whether another ODT excipient is necessary. Ohrem and Ognibene, Pharmaceutical Technology Europe 21 (9) Sep. 1, 2009.

Nishizawa et al. disclose rapidly disintegrating tablet in the oral cavity. EP 1 944 017 A2.

### SUMMARY OF THE INVENTION

The invention broadly regards ODTs having low levels of superdisintegrant and optionally having at least one active pharmaceutical or nutraceutical ingredient (API). More particularly, the invention comprises an ODT having about 0.3% to 2% (wt/wt) sodium croscarmellose, a polyol(s) matrix, optionally a lubricant, and optionally an API, which ODT disintegrates in water within 30 sec and has a friability less than 0.5%.

In one aspect, the invention comprises an aged direct compression ODT comprising or consisting essentially of between about 0.3% to about 2% (wt/wt) sodium croscarmellose relative to the total weight of the ODT, a polyol matrix, optionally a lubricant, and optionally an API, wherein after storage for four months a tensile strength of the ODT is at least 0.5 MPa and a disintegration time using an excess water test is less than 30 seconds.

In another aspect, the invention comprises a direct compression ODT, consisting essentially of about 0.3% (wt/wt) to 2.0% (wt/wt) sodium croscarmellose relative to the total weight of the ODT, from about 0.1% (wt/wt) to about 2% magnesium stearate, optionally from 0.1% to less than 10% (wt/wt) microcrystalline cellulose, up to about 20 (wt/wt) of an API, optionally one or more colorants, sweeteners, fragrances, flavor compounds, and/or flavor blockers, and the balance spray-dried mannitol, a) wherein the dry ODT has a tensile strength from 0.5 to 1.7 MPa, b) wherein the disintegration time in excess water is less than 30 seconds, and c) wherein the friability is less than 1.0%, preferably less than 0.5%.

In yet another aspect, the invention comprises from about 0.3% (wt/wt) to 2.0% (wt/wt) superdisintegrant relative to the total weight of the ODT, a lubricant, an API, optionally one or more colorants, sweeteners, fragrances, flavor compounds, and/or flavor blockers, and the balance spray-dried polyol, a) wherein the dry ODT has a tensile strength from 0.3 to 1.7 MPa, b) wherein the initiation of disintegration using a small volume texture pressure test in 2 mL of water is less than 20 seconds, and c) wherein the friability is less than 0.5%.

### BRIEF DESCRIPTION OF THE FIGURES

FIG. 1. Evaluation of aspirin ODTs.

FIG. 2. Evaluation of acetaminophen ODTs.

FIG. 3. Evaluation of naproxen ODTs.

FIG. 4. Evaluation of theophylline ODTs.

FIG. 5. Evaluation of chlorpheniramine maleate ODTs.

FIG. 6. Disintegration of an aged ODT having acetaminophen.

FIG. 7. ODT disintegration process in a small volume as demonstrated by a texture analyzer test.

### DETAILED DESCRIPTION

Further disclosure is provided below.

By poor solubility in water is meant that less than or equal to 10 mg of the ingredient is soluble in one mL neutral distilled water at 25° C. Thus theophylline, having a solubility of about 8 mg/mL is poorly soluble in water, as the term is used herein. Similarly, naproxen, having a solubility of less than 1 mg/mL (reported as 0.07 mg/mL) is also poorly soluble in water. The absence or presence of a salt form can substantially change solubility. For example, chlorphenamine is poorly soluble in water, yet chlorphenamine maleate is very soluble.

By the term “about” when referring to a value, is meant specifically that a measurement can be rounded to the value using a standard convention for rounding numbers. For example, “about 1.5” is 1.45 to 1.54.

By the term “less than about” when referring to a value, is meant specifically that the value does not exceed upper range of the value. For example, “less than about 1.5” is less than 1.54.

By the term “aged” regarding an ODT is meant that the ODT is kept in a sealed container at room temperature for a period of more than two weeks. A specific term of aging can be specified, for example including 2 months, 3 months, 4 months, 5 months, or a range, for example including 3-12 months, each calculated from the date of making the ODT. A preferred period for evaluating the effect of aging on ODTs is four months.

The inventors have shown that ODTs can be prepared that disintegrate quickly in the mouth and yet have relatively low proportions of superdisintegrant, typically less than about 2% (wt/wt), and have no inorganic excipients. The ODTs have excellent hardness and low friability. Moreover, the ODTs dissolve smoothly and leave no gritty sensation. That is, they have excellent mouth feel. These ODTs are obtainable by using specific narrow ranges of particular ingredients. Thus, the ODT compositions disintegrate in the mouth in less than 30 seconds, preferably less than 20 seconds, once they come into contact with saliva in the oral cavity, and which are hardly noticed on the tongue. In one embodiment, the ODT has about 0.5% (wt/wt) superdisintegrant. In another embodiment, the ODT has about 0.8% (wt/wt) superdisintegrant. In yet another embodiment, the ODT has about 1.0% (wt/wt) superdisintegrant. In still another embodiment, the ODT has about 1.2% (wt/wt) superdisintegrant. The ODT can have about 1.4% (wt/wt) superdisintegrant. In one embodiment, the ODT has about 1.6% (wt/wt) superdisintegrant. In another embodiment, the ODT has about 1.8% (wt/wt) superdisintegrant. In yet another embodiment, the ODT has about 2.0% (wt/wt) superdisintegrant. In still another embodiment, the ODT has between about 0.5% and about 0.9% (wt/wt) superdisintegrant. In still another embodiment, the ODT has between about 1.0% and about 1.4% (wt/wt) superdisintegrant. In still another embodiment, the ODT has between about 1.5% and about 1.9% (wt/wt) superdisintegrant. The superdisintegrant can be croscarmellose, crospovidone, or SSG, or a combination thereof. In a preferred embodiment, the superdisintegrant is croscarmellose.

The ODT has a matrix that binds the ingredients together while in the solid form. Advantageously, the matrix disintegrates rapidly and smoothly in the mouth, without any sensation of astringency, off-flavor, or grittiness. Other advantageous features of matrix materials are high aqueous solubility, high compressibility, sweetness, and a negative heat of solution. The matrix should be a solid at room temperature. Several matrix materials are known for tablets, including: dextrose, erythritol, fructose, isomalt, lactilol, maltilol, maltose, mannitol, sorbitol, starch hydrolysate, polydextrose, and xylitol. Polyols, that is, sugar alcohols, are advantageous and can include xylitol, lactitol, mannitol, sorbitol, and maltitol. Spray dried sorbitol and gamma-crystalline sorbitol are available. Spray-dried mannitol is a preferred matrix.

Alternatively, the matrix can be a combination of constituents. One combination is spray-dried mannitol and microcrystalline cellulose. The matrix can comprise 100% spray-dried mannitol, 100% microcrystalline cellulose, and any intermediate combination of the mannitol and the cellulose. In one embodiment, the matrix is up to 50% microcrystalline cellulose and the balance is spray dried mannitol. In another embodiment, the matrix is up to 25% microcrystalline cellulose and the balance is spray dried mannitol. In yet another embodiment, the matrix is up to 10% microcrystalline cellulose and the balance is spray dried mannitol. In other embodiments, the matrix can be up to about 8%, up to about 6%, or up to about 4% microcrystalline cellulose. The microcrystalline cellulose can be, for example, Avicel® PH 101, Avicel® PH 102, Avicel® HFE 102, or Emcocel® 50 M, which have an average particle size of less than approximately 100 μm, and 99% by weight is below 250 μm. In one embodiment, the ODT lacks microcrystalline cellulose. Another matrix is a combination of spray dried mannitol and xylitol.

In one embodiment, the matrix is a single component. In another embodiment, the matrix lacks at least one of dextrose, erythritol, fructose, isomalt, lactilol, maltilol, maltose, sorbitol, starch hydrolysate, polydextrose, and xylitol.

Spray dried mannitol is an excellent matrix for the ODT. Spray dried mannitol is commercially available: Pearlitol® SD (Roquette) and Mannogen™ EZ spray dried mannitol (SPI Pharma). Spray dried mannitol has several useful physical or chemical properties. For example, it dissolves easily in water (1 in 5.5 parts at 20° C.) and quickly (5 g dissolves in approximately 5 sec in 150 mL of water at 20° C.) Direct compression mannitol, powder mannitol, and other related saccharide excipients are slower to dissolve. Spray dried mannitol is substantially in the α crystalline form, whereas other forms of mannitol are generally in the β form. Moreover, spray dried mannitol has flowability of 6 seconds, which is desirable for direct compression processes. It is highly compressible, having a Cohesion Index of 1500-2000. It also has good dilution capacity due to the size and form of the particle, which makes it possible to accept large amounts of active ingredients that are not easily compressed. Spray dried mannitol is very chemically stable, is non-hygroscopic, and does not form Maillard reactions with amino groups. Moreover, consumers experience a sense of freshness when taking mannitol because of its negative heat of dissolution. Spray dried mannitol has about half the sweetness of sucrose. It is also very palatability because of its small particle size.

In one embodiment, the spray-dried mannitol comprises the balance of the ODT.

There are no limitations to the API used in these ODTs. However, active ingredients useful for patients with swallowing difficulties are preferred. The ODT can comprise a combination of APIs.

More particularly, API can include, but are not limited to: analgesics: acetaminophen, aspirin, naproxen; anti-ulcer drugs: famotidine; antiemetics: ondansetron, granisetron, dolasetron, domperidone, metoclopramide; antihypertensive drugs: enalapril, losartan, candesartan, valsartan, lisinopril, ramipril, doxazosin, terazosin; antihistaminic drugs: loratadine, cetirizine; antipsychotic drugs: risperidone, olanzapine, quetiapine; antidepressants: paroxetine, fluoxetine, mirtazapine; analgesics and anti-inflammatory drugs: piroxicam; antihypercholesterolemic drugs: simvastatin, lovastatin, pravastatin; antimigraine drugs: zolmitriptan, naratriptan, rizatriptan; anti-epileptic drugs: lamotrigine; anti-Parkinson drugs: selegiline, apomorphine; anxiolytic drugs: diazepam, lorazepam, zolpidem; anti-asthma drugs: zafirlukast, montelukast; erection dysfunction agents: sildenafil; both in their free base form and in their acceptable pharmaceutical salts, hydrates, solvates or isomers. An API can also be one or more of alprazolam, prednisilone, zomitriptan, selegiline, baclofen, carbidopa, levodopa, desloratadine, aripiprazole, loratadine, or donepezil.

Preferably, the API comprises about 50% (wt/wt) of the ODT or less. The API can be about 40% (wt/wt) of the ODT. In one embodiment the API is about 30% (wt/wt) of the ODT. More preferably, the API comprises 20% or less than about 20% (wt/wt). In another embodiment, the API content is at least 0.01% (wt/wt). In yet another embodiment, the API content is less than about 12% (wt/wt), less than 10% (wt/wt), less than 8% (wt/wt), less than 6% (wt/wt), less than 4% (wt/wt), or less than 2% (wt/wt). The actual amount of the API will reflect the usual dosage or some integral fraction thereof. Also, the API is preferably a fine powder, where at least 90% by weight of the API has a particle size of below 100 μm.

In one embodiment, the ODT can have an API selected from the group consisting of an acidic poorly water soluble ingredient, a basic poorly water soluble ingredient, an acidic water soluble ingredient, and a basic water soluble ingredient.

The ODT can have a disintegration time of less than about 60 seconds (sec), preferably less than about 50 sec, preferably less than about 40 sec, preferably less than about 30 sec, more preferably less than about 20 sec, and yet more preferably less than about 15 sec. In one embodiment, the disintegration time is between about 10 sec and about 30 sec. In another embodiment, the disintegration time is less than 30 sec. In a preferred embodiment, the disintegration time is less than 20 seconds.

The ODT can have a hardness of greater than 20 N or greater than 30 N. Preferably, the ODT has a hardness value greater than 40 N. The hardness can exceed 50 N. The ODT can have a hardness value greater than 60 N. In one embodiment, the hardness of the ODT is between 40 N and about 150 N. In general, the measured hardness is a function of the size and shape of the ODT. On the other hand, tensile strength is independent of the size and shape of the ODT.

The ODT can have a tensile strength of greater than about 0.3 MPa. In a preferred embodiment, the tensile strength is greater than 0.5 MPa. In yet another embodiment, the tensile strength is greater than 0.6 MPa. In still another embodiment, the tensile strength is greater than 0.7 MPa. In still yet another embodiment, the tensile strength is greater than 0.8 MPa. In one embodiment, the tensile strength is greater than 0.9 MPa. In another embodiment, the tensile strength is greater than 1.0 MPa. In yet another embodiment, the tensile strength is greater than 1.1 MPa. In still yet another embodiment, the tensile strength is greater than 1.2 MPa. The tensile strength can be greater than 1.3 MPa. In one embodiment, the tensile strength is greater than 1.5 MPa. The tensile strength can be less than 1.7 MPa. In one embodiment the tensile strength is between 0.55 MPa and 1.7 MPa. In another embodiment, the tensile strength is between 1.0 MPa and 1.5 MPa.

The ODT can have a friability of less than 1%. In a preferred embodiment, the friability is less than 0.8%. In another embodiment, the friability is less than 0.6%. In a more preferred embodiment, the friability is less than 0.5%. However, the friability can advantageously be less than 0.4% or 0.2%. These friability values enable packaging in any kind of package using conventional machinery, and do not require any special care to be taken in the intermediate bulk storage of the ODTs or in the feed systems used in the packaging operation.

The ODTs can further comprising one or more colorants, sweeteners, fragrances, flavor compounds, flavor blockers, and/or additional disintegrants. In the aggregate, the colorants, sweeteners, fragrances, flavor compounds, and chemical flavor blockers (which do not include mannitol and the API) comprise less than 6% (wt/wt) of the ODT, preferably less than 5%, more preferably less than 4%, and most preferably less than 3%.

The ODT can comprise one or more colorants. Any pharmaceutically acceptable colorant is useful. Moreover, the colorants may include pigments, natural food colors and dyes suitable for food, drug and cosmetic applications. A full recitation of all F.D. & C. colorants and their corresponding chemical structures can be found in the Kirk-Othmer Encyclopedia of Chemical Technology, 3rd Edition, in volume 5 at pages 857-884, of which text is incorporated herein by reference. In one embodiment, the ODT lacks colorant.

In addition to any sweet sensation provided by the matrix, the ODT can comprise one or more natural or artificial sweetners. Sweetener means a compound other than matrix or API. Any pharmaceutically acceptable sweetner is useful. The following sweetners may be used, but the list does not exclude other options: aspartame, sodium cyclamate, sodium saccharine, ammonium glycyrrhizinate, neohesperidine dihydrochalcone, alitame, neotame, sucralose, stevioside, sucrose, fructose, lactose, sorbitol, and xylitol. In one embodiment, the ODT lacks sweetener.

The ODT can comprise one or more fragrances. Any pharmaceutically acceptable fragrance may be used, including floral, fruity, woody, and fougere fragrances. In one embodiment, the ODT lacks a fragrance compound.

The ODT can comprise one or more natural or artificial flavor compounds, including those having flavors like menthol, mint, or fruit. Flavors such as raspberry, blackberry, cherry, black cherry, black currant, strawberry, grape, lingonberry, cantaloupe, watermelon, pear, apple, pineapple, mango, peach, apricot, plum, orange, lemon, lime, spearmint, peppermint, vanilla, and chocolate are suitable. Other flavors can include the flavor of bubblegum. The flavor compound can encompass a flavor enhancer, e.g. citric acid. The flavor compound is typically less than 1.5% (wt/wt), preferably about 0.75% to 0.5%. A flavor enhancer is typically less than 1.5% (wt/wt), preferably about 0.08%.

It is well known by those skilled in the art to employ physical and chemical means to provide immediate or controlled release of API within desired portions in the gastrointestinal tract. These means include tastemasking to avoid dissolution of a poor tasting API in the mouth or to prevent or inhibit contact of the poor tasting API with taste buds in the oral cavity. Tastemasking includes but is not limited to complexing, granulating, encapsulating, layering, or coating of the API or otherwise minimizing contact of the poor tasting API with saliva and taste buds. In one embodiment, the API can be in the form of tastemasked particulates which provide acceptable taste and mouthfeel in the ODT. In one embodiment, the API is released in the oral cavity. In another embodiment, the API is not released in the oral cavity.

The ODT can comprise a chemical flavor blocker. Flavor blockers that moderate bitter or medicinal flavors are useful for an API that has a bitter or medicinal flavor. Any flavor blocker can be used, depending on the flavor to be masked. Among suitable flavor blockers are cream flavor. Also useful for blocking unpleasant tasting flavors is a combination of aspartame, ammonium glycyrrhizinate, mentholated flavoring and L-menthol (0.1-0.2% in weight). These agents have a refreshing effect that can support the effect of mannitol. Use of thaumatin is envisioned to provide sweetness with a prolonged licorice-like taste.

Making the ODT without a lubricant or with a lubricant is envisioned. In one embodiment, the ODT has a lubricant. Those of skill in the art can select a suitable lubricant from those known in the art. Stearic acid and polyethylene glycol (M_{R}>2000) are known, relatively hydrophilic, lubricants. Sodium stearyl fumarate is a suitable lubricant. Magnesium stearate has preferred properties. The amount of lubricant can be less than about 10% (wt/wt), preferably less than about 5% (wt/wt), and more preferably about 3% (wt/wt), about 2% (wt/wt), about 1% (wt/wt), or about 0.1% (wt/wt), or any intermediate value. The range of 0.1% to about 1.5% is preferred for magnesium stearate. More preferred are 0.1%, 0.2%, 0.3%, 0.4%, 0.5%, 0.6%, 0.7%, 0.8%, 0.9%, 1.0%, 1.1%, 1.2%, 1.3%, 1.4%, or 1.5% magnesium stearate, all by weight.

A lubricant can have effects in addition to lubrication. These other effects can include a different uptake or a different rate of uptake of moisture or fluid. Effects of the lubricant may relate to the hydrophobicity of a lubricant.

In one embodiment, the ODT has one or more other components, which may be selected from a mold release agent and a second disintegrant. The second disintegrant can be microcrystalline cellulose. In one embodiment, the ODT lacks disintegrants other than polyol matrix and croscarmellose. In one embodiment, the ODT lacks disintegration enhancers such as silicon dioxide.

Any weight of the ODT is acceptable, if acceptable to consumers. In one embodiment, the ODT weighs more than 40 mg. The ODT can weigh more than 60 mg, more than 80 mg, more than 100 mg, more than 200 mg, or more than 300 mg.

The following examples illustrate embodiments of the invention but do not limit its scope.

### EXAMPLES

### Example 1

### Materials

Spray-dried mannitol was obtained as Pearlitol® 200 SD from Roquette (Paris, France) which is a direct compressible mannitol, and was used as an ODT matrix. Magnesium stearate (Mallinckrodt, Hazelwood, Mo.) was used as a lubricant.

Table 1 shows three commercial superdisintegrants available from four suppliers. Lot TN07817522 of Ac-Di-Sol, which has a D50 of 42.5, was used in the examples, except that lot TN09820342 was used in the small ODT and flavor examples. Other gradesand types of superdisintegrants can be used, which differ in physical properties, such as particle size.

TABLE 1
Commercial superdisintegrants

| Category | Commercial name | Supplier |
|---|---|---|
| Croscarmellose | Ac-Di-Sol ® | FMC Corporation |
| Crospovidone | Polyplasdone ® XL-10 (PVP XL-10) | ISP, Inc. |
| | Kollidon ® CL-SF | BASF |
| Sodium Starch | Glycolys ® | Roquette |
| Glycolate | | |

### Example 2

### Preparation of ODTs

To prepare each formulation, Pearlitol® 200 SD and disintegrant, were weighed and premixed in a V-blender for 15 minutes; then magnesium stearate was added and followed up with additional 2 minutes of mixing. The ODTs had the specified amount of superdisintegrant, 1.5% (wt/wt) magnesium stearate, and the balance spray dried mannitol.

To prepare ODTs, each formulation was compressed individually on a Stokes 512 Tablet Press with four stations. Standard 7/16″ concave punches and corresponding dies were used. ODT weight was adjusted to 400 mg. SMI Director™ data acquisition system was used to record the compaction process. Compaction forces of 4 kN, 6 kN, 8 kN, 10 kN, or 12 kN were applied to the formulations to produce ODTs with different hardness.

### Example 3

### Characterization of ODTs

Disintegration times of ODTs were determined using a Hanson QC-21 disintegration test system. The test was conducted at 37±0.5° C. in a medium of 10 mL distilled water. Six ODTs per sample were analyzed and the mean is reported.

Hardness along with ODT weight, thickness, and diameter were determined using an AT4 automatic tablet-testing system (Dr. Schleuniger Pharmatron, Switzerland). The hardness data are reported as the mean hardness of ten individual determinations. ODT weight and thickness were controlled in a very tight range.

ODT friability was measured on a VanKel Friabilator rotated at 25 rpm for 5 minutes. Twenty ODTs per sample were randomly selected for the study. The friability for each sample was calculated using following equation:

Friability (%)=(W_{b}−Wₐ)/W_{b}×100

where W_{b} and Wₐ are the weights before and after friability test.

All initial ODT characterization studies (hardness, disintegration time, and friability) were performed on ODTs that were stored for 24 hours at ambient condition in closed plastic bags. For evaluation of the effect of aging, ODTs were stored at ambient condition in closed plastic bags for 4 months, and then hardness, disintegration time, and friability were re-evaluated using the same procedures as in the initial studies.

### Mouth Feel of ODTs

Mouth feel was determined with a triangle test. In general, a triangle test is a difference test having an internal control to determine the rigor of a sensory difference between two products. Here, the triangle mouth feel test was used to determine if there is any mouth feel difference between ODTs that contain 2% croscarmellose and ODTs that contain 5% PVP XL-10.

In each set of study, a set of three coded ODT samples were presented to one panelist. Within the three coded samples, two were identical and one was different. For example, the three samples could be either two identical ODTs that contained 2% croscarmellose plus one ODT that contained 5% PVP XL-10; or two identical ODTs that contained 5% PVP XL-10 plus one ODT that contained 2% croscarmellose. The panelists were asked to pick out the one sample they felt was different from the other two in terms of mouth feel. The perceived difference may come from the disintegration time, taste, texture, etc. and each panelist was asked to describe it in the comments section. A total of 34 ODT sample sets (102 ODTs) were tested, using ODTs that were compressed at 8 kN. All judgments were collected and used to draw a statistical conclusion.

### Texture Analyzer Evaluation of ODT Disintegration in Shallow Water

A TA-XT2i texture analyzer was used to monitor the disintegration process of ODTs in shallow water. The settings were “measure distance in compression, hold until time,” with a 5 kg load cell, ½″ diameter clear cylindrical probe. The automatic surface detection trigger was set to 1 gram, with a test speed of 0.1 mm/second. For each test, a small weighing boat with 2 mL of deionized water was placed under the probe with a height set at 7 mm. The ODT to be tested was put into the 2 mL water and the test started simultaneously.

### Example 4

### Preparation and Characterization of ODTs

Each formulation was prepared as above.

Features of ODTs having a 0.5% (wt/wt) disintegrant level in ODTs made of disintegrant, magnesium stearate, and mannitol are shown in Table 2.

In the tables 2-5 and 7-8, where used, “force” is the compaction force used in ODT preparation; “weight” which is the mass, “thickness,” and “diameter” are physical properties of the finished ODT; “hardness” is the ODT crushing strength; “tensile strength” is calculated from the hardness, shape, and the dimensions of the ODT; and “disintegration time” is as measured by the excess water test, above. Where provided, the error measurements are standard deviations.

TABLE 2

| | | | | | | Tensile | | |
|---|---|---|---|---|---|---|---|---|
| Disintegrant | Force | Weight | Thickness | Diameter | Hardness | Strength | Disintegration | Friability |
| (0.5%) | (kN) | (mg) | (mm) | (mm) | (N) | (MPa) | time (sec) | (%) |
| Ac-Di-Sol ® | 4.2 | 402.8 | 5.02 | 11.17 | 26.40 ± 0.97 | 0.347 | 11.7 ± 6.3 | 0.65 |
| Ac-Di-Sol ® | 6.0 | 402.1 | 4.83 | 11.18 | 40.70 ± 1.70 | 0.558 | 17.6 ± 0.5 | 0.31 |
| Ac-Di-Sol ® | 8.1 | 402.7 | 4.68 | 11.19 | 61.20 ± 2.82 | 0.870 | 26.1 ± 1.1 | 0.39 |
| Ac-Di-Sol ® | 10.2 | 403.3 | 4.57 | 11.20 | 81.89 ± 2.15 | 1.196 | 36.1 ± 1.9 | 0.31 |
| Ac-Di-Sol ® | 12.1 | 403.5 | 4.49 | 11.2 | 97.70 ± 2.54 | 1.459 | 45 ± 3.8 | 0.29 |
| Kollidon ® CL | 4.2 | 399.2 | 4.89 | 10.89 | 24.3 | 0.337 | 130.6 | |
| Kollidon ® CL | 6.0 | 399.1 | 4.65 | 10.90 | 37.5 | 0.552 | 235.5 | |
| Kollidon ® CL | 8.2 | 401.9 | 4.53 | 10.91 | 64.5 | 0.978 | 232.9 | |
| Kollidon ® CL | 10.0 | 401.0 | 4.52 | 10.90 | 73.6 | 1.120 | 171.05 | |
| Kollidon ® CL | 12.1 | 400.1 | 4.39 | 10.91 | 98.2 | 1.545 | 191.6 | |
| Kollidon ® CL-F | 4.1 | 399.8 | 5.02 | 10.90 | 23.7 | 0.319 | 165 | |
| Kollidon ® CL-F | 6.1 | 401.4 | 4.83 | 10.90 | 39.4 | 0.554 | 262.6 | |
| Kollidon ® CL-F | 8.1 | 401.5 | 4.64 | 10.91 | 60.0 | 0.884 | 246.7 | |
| Kollidon ® CL-F | 10.2 | 400.3 | 4.50 | 10.91 | 80.2 | 1.225 | 225 | |
| Kollidon ® CL-F | 12.2 | 402.1 | 4.44 | 10.92 | 97.0 | 1.506 | 187.4 | |
| Kollidon ® CL-SF | 4.1 | 405.0 | 5.04 | 10.89 | 28.7 | 0.384 | 242.6 | |
| Kollidon ® CL-SF | 6.0 | 401.9 | 4.79 | 10.90 | 45.0 | 0.639 | 279.2 | |
| Kollidon ® CL-SF | 8.1 | 402.1 | 4.66 | 10.92 | 66.5 | 0.973 | 226.9 | |
| Kollidon ® CL-SF | 10.1 | 404.7 | 4.55 | 10.89 | 88.1 | 1.332 | 232.4 | |
| Kollidon ® CL-SF | 12.1 | 402.2 | 4.47 | 10.90 | 101.3 | 1.561 | 185.1 | |
| Glycolys ® | 4.0 | 401.5 | 5.14 | 11.15 | 27.0 | 0.346 | 18.8 | |
| Glycolys ® | 6.0 | 400.3 | 4.86 | 11.15 | 49.7 | 0.679 | 32.1 | |
| Glycolys ® | 8.1 | 398.4 | 4.67 | 11.15 | 72.3 | 1.035 | 40.7 | |
| Glycolys ® | 10.1 | 401.4 | 4.55 | 11.16 | 103.0 | 1.519 | 51.6 | |
| Glycolys ® | 12.1 | 403.8 | 4.49 | 11.17 | 129.6 | 1.937 | 70.8 | |
| Polyplasdone ® | 4.1 | 397.8 | 5.01 | 10.90 | 23.4 | 0.317 | 123.1 | |
| XL | | | | | | | | |
| Polyplasdone ® | 6.1 | 396.4 | 4.75 | 10.9 | 39.34 | 0.565 | 201.4 | |
| XL | | | | | | | | |
| Polyplasdone ® | 8.1 | 395.4 | 4.65 | 10.9 | 57.49 | 0.846 | 224.5 | |
| XL | | | | | | | | |
| Polyplasdone ® | 10.1 | 397.6 | 4.51 | 10.9 | 74.75 | 1.139 | 186.6 | |
| XL | | | | | | | | |
| Polyplasdone ® | 12.1 | 397.8 | 4.46 | 10.9 | 88.58 | 1.364 | 186.4 | |
| XL | | | | | | | | |

Features of ODTs having a 1.0% (wt/wt) disintegrant level in ODTs made of disintegrant, magnesium stearate, and mannitol are shown in Table 3.

TABLE 3

| | | | | | | Tensile | Disintegration | |
|---|---|---|---|---|---|---|---|---|
| Disintegrant | Force | Weight | Thickness | Diameter | Hardness | Strength | time | Friability |
| (1.0%) | (kN) | (mg) | (mm) | (mm) | (N) | (MPa) | (sec) | (%) |
| Ac-Di-Sol ® | 4.1 | 397.7 | 4.98 | 11.19 | 24.80 ± 1.81 | 0.328 | 11.4 ± 0.5 | 0.59 |
| Ac-Di-Sol ® | 6.1 | 397.1 | 4.80 | 11.18 | 41.20 ± 2.35 | 0.569 | 13.5 ± 0.33 | 0.36 |
| Ac-Di-Sol ® | 8.1 | 398.7 | 4.68 | 11.18 | 61.00 ± 2.49 | 0.869 | 18 ± 0.54 | 0.34 |
| Ac-Di-Sol ® | 10.1 | 402.0 | 4.60 | 11.18 | 76.50 ± 2.84 | 1.111 | 26.3 ± 0.9 | 0.21 |
| Ac-Di-Sol ® | 12.1 | 401.4 | 4.50 | 11.2 | 95.80 ± 2.25 | 1.425 | 31.5 ± 1.1 | 0.23 |
| Glycolys ® | 4.0 | 402.0 | 5.13 | 11.14 | 27.1 | 0.348 | 17.1 | 1.02 |
| Glycolys ® | 6.0 | 400.8 | 4.86 | 11.15 | 46.5 | 0.635 | 22.4 | 0.52 |
| Glycolys ® | 8.1 | 400.5 | 4.70 | 11.16 | 72.8 | 1.033 | 30.3 | 0.42 |
| Glycolys ® | 10.1 | 399.8 | 4.57 | 11.17 | 95.0 | 1.393 | 40.5 | 0.27 |
| Glycolys ® | 12.1 | 400.4 | 4.48 | 11.16 | 121.4 | 1.823 | 51.1 | 0.29 |
| Kollidon ® CL- | 4.0 | 401.4 | 5.03 | 10.88 | 26.5 | 0.357 | 168.4 | 0.72 |
| SF | | | | | | | | |
| Kollidon ® CL- | 6.1 | 403.1 | 4.84 | 10.88 | 45.9 | 0.646 | 192.3 | 0.43 |
| SF | | | | | | | | |
| Kollidon ® CL- | 8.1 | 403.5 | 4.64 | 10.87 | 68.0 | 1.006 | 221.1 | 0.36 |
| SF | | | | | | | | |
| Kollidon ® CL- | 10.1 | 402.4 | 4.55 | 10.89 | 88.3 | 1.334 | 184.8 | 0.27 |
| SF | | | | | | | | |
| Kollidon ® CL- | 12.2 | 403.3 | 4.47 | 10.91 | 111.7 | 1.720 | 181.7 | 0.29 |
| SF | | | | | | | | |
| Polyplasdone ® | 4.0 | 400.9 | 5.10 | 11.2 | 23.20 | 0.300 | 111.4 | 0.88 |
| XL10 | | | | | | | | |
| Polyplasdone ® | 6.1 | 402.6 | 4.87 | 11.1 | 44.10 | 0.601 | 191.7 | 0.55 |
| XL10 | | | | | | | | |
| Polyplasdone ® | 8.1 | 401.7 | 4.70 | 11.2 | 62.50 | 0.866 | 248.2 | 0.43 |
| XL10 | | | | | | | | |
| Polyplasdone ® | 10.1 | 400.9 | 4.59 | 11.2 | 80.60 | 1.178 | 217.5 | 0.28 |
| XL10 | | | | | | | | |
| Polyplasdone ® | 12.1 | 400.5 | 4.49 | 11.2 | 99.50 | 1.492 | 216.1 | 0.30 |
| XL10 | | | | | | | | |

Features of ODTs having a 1.5% (wt/wt) croscarmellose level in ODTs made of disintegrant, magnesium stearate, and mannitol are shown in Table 4.

TABLE 4

| | | | | | | Tensile | | |
|---|---|---|---|---|---|---|---|---|
| Disintegrant | Force | Weight | Thickness | Diameter | Hardness | Strength | Disintegration | Friability |
| (1.5%) | (kN) | (mg) | (mm) | (mm) | (N) | (MPa) | time (sec) | (%) |
| Ac-Di-Sol ® | 4.0 | 400.5 | 5.01 | 11.13 | 18.80 ± 0.63 | 0.248 | 11.7 ± 0.3 | 0.94 |
| Ac-Di-Sol ® | 6.1 | 400.2 | 4.76 | 11.14 | 33.30 ± 1.06 | 0.467 | 12.9 ± 0.3 | 0.53 |
| Ac-Di-Sol ® | 8.1 | 399.2 | 4.60 | 11.13 | 49.90 ± 1.52 | 0.728 | 15 ± 0.5 | 0.43 |
| Ac-Di-Sol ® | 10.1 | 400.2 | 4.50 | 11.14 | 66.90 ± 2.64 | 1.001 | 18 ± 0.4 | 0.37 |
| Ac-Di-Sol ® | 12.2 | 399.5 | 4.39 | 11.1 | 85.40 ± 3.10 | 1.315 | 24.3 ± 1.1 | 0.30 |

Features of ODTs having a 2.0% (wt/wt) disintegrant level in ODTs made of disintegrant, magnesium stearate, and mannitol are shown in Table 5.

TABLE 5

| | | | | | | Tensile | | |
|---|---|---|---|---|---|---|---|---|
| Disintegrant | Force | Weight | Thickness | Diameter | Hardness | Strength | Disintegration | Friability |
| (2%) | (kN) | (mg) | (mm) | (mm) | (N) | (MPa) | time (sec) | (%) |
| Ac-Di-Sol ® | 3.9 | 400.9 | 5.13 | 11.17 | 25.10 | 0.322 | 11.7 | 0.85 |
| Ac-Di-Sol ® | 6.1 | 400.6 | 4.86 | 11.17 | 48.10 | 0.656 | 13.6 | 0.50 |
| Ac-Di-Sol ® | 8.2 | 401.4 | 4.70 | 11.18 | 73.60 | 1.041 | 15 | 0.33 |
| Ac-Di-Sol ® | 10.2 | 401.1 | 4.60 | 11.17 | 96.60 | 1.403 | 18.1 | 0.29 |
| Ac-Di-Sol ® | 12.2 | 401.6 | 4.50 | 11.2 | 114.70 | 1.708 | 22.9 | 0.23 |
| Kollidon ® CL-SF | 4.0 | 399.1 | 5.10 | 10.90 | 24.4 | 0.322 | 8.8 | |
| Kollidon ® CL-SF | 6.1 | 397.9 | 4.81 | 10.96 | 45.2 | 0.635 | 12.1 | |
| Kollidon ® CL-SF | 8.2 | 398.0 | 4.64 | 10.89 | 67.9 | 1.002 | 19 | |
| Kollidon ® CL-SF | 10.2 | 400.0 | 4.56 | 10.91 | 91.6 | 1.377 | 26.8 | |
| Kollidon ® CL-SF | 12.2 | 399.9 | 4.47 | 10.98 | 113.6 | 1.740 | 46.8 | |
| Glycolys ® | 4.0 | 399.3 | 5.13 | 11.14 | 25.4 | 0.326 | 16 | |
| Glycolys ® | 6.1 | 401.3 | 4.87 | 11.15 | 47.3 | 0.645 | 20.2 | |
| Glycolys ® | 8.1 | 400.5 | 4.69 | 11.16 | 70.7 | 1.005 | 25.6 | |
| Glycolys ® | 10.1 | 399.6 | 4.54 | 11.17 | 91.9 | 1.357 | 32.5 | |
| Glycolys ® | 12.1 | 401.1 | 4.48 | 11.17 | 117.8 | 1.769 | 39.9 | |
| Polyplasdone ® | 4.0 | 399.9 | 5.11 | 11.2 | 21.40 | 0.276 | 13 | |
| XL | | | | | | | | |
| Polyplasdone ® | 6.1 | 400.6 | 4.86 | 11.2 | 39.50 | 0.540 | 20.2 | |
| XL | | | | | | | | |
| Polyplasdone ® | 8.0 | 404.0 | 4.73 | 11.2 | 60.50 | 0.853 | 24.7 | |
| XL | | | | | | | | |
| Polyplasdone ® | 10.0 | 402.4 | 4.61 | 11.2 | 70.60 | 1.024 | 36.3 | |
| XL | | | | | | | | |
| Polyplasdone ® | 12.0 | 402.2 | 4.52 | 11.2 | 100.70 | 1.498 | 60.9 | |
| XL | | | | | | | | |

Characterization of ODTs

The Tables 2-5 show ODT disintegration times for ODTs made with different superdisintegrants, with different amounts of disintegrants, and different compaction forces. Different tables represent different disintegrant use levels. Additional studies using higher disintegrant concentrations showed that high levels decreased disintegrant function, particularly for croscarmellose. The results indicate that croscarmellose ODTs disintegrate much faster than crospovidone and SSG at low use level (≦2%). Crospovidone starts to be effective at 5% and higher amounts. SSG is less potent than croscarmellose at low use level and less potent than crospovidone at high level.

In fact, at the lowest use level (0.5%), croscarmellose was the only disintegrant among the three that can effectively disintegrate ODTs at all compaction forces in the range. Even when the use level increased to 2%, croscarmellose was still the only disintegrant that could provide ODTs which meet the USP required 30 second disintegration time, at all compaction force ranges (Table 5).

Tables 2-5 also show ODT hardness for all disintegrants at different use levels and compaction forces (4 kN-12 kN). Results showed that disintegrants had little impact on hardness when the disintegrant use level was ≦5%, as the hardness was similar to control sample at all compaction force range. However, hardness started to decrease when the use level was ≧8%, and ODTs became softer. In addition, the ODT friability data further confirmed the hardness conclusions, e.g., ODTs became more friable if disintegrant use level reached or exceeded 8%.

For each disintegrant, an optimal use level, which means the lowest use level that can achieve its fastest disintegration time over the entire studied compaction force ranges, was identified. The optimal use level for croscarmellose is 2%, and for the others is 5%. Detailed data are not shown here. In general, 2% croscarmellose could disintegrate ODTs at the same fast speed as 5% crospovidone, and both outperformed 5% SSG.

### In Vivo Mouth Feel Study of ODTs

In terms of mouth feel of ODTs that contain 2% croscarmellose vs. ODTs that contain 5% PVP XL-10, 15 out of the 34 panelists provided the correct judgment. This means 15 panelists picked out the correct odd sample, while the other 19 panelists picked out one of the two identical samples instead of the odd one. According to a statistical table by Meilgaard et al. Sensory Evaluation Techniques, 3^{rd} edition, CRC Press LLC, Boca Raton, Fla., New York, N.Y. 1999, pp. 369, a minimum of 17 correct judgments would be required to establish significance at 95% probability level. A minimum of 19 correct judgments would be required to establish significance at 99% probability level. Thus, the conclusion from this triangle mouth feel study is that there is no significant statistical difference between ODTs that contain 2% croscarmellose and ODTs that contain 5% PVP XL-10 in terms of mouth feel.

### Example 5

### Features of ODTs having API

### Characterization of ODTs

For ODTs having an API, physical properties, such as hardness, weight, thickness, and diameter, were determined using an AT4 automatic tablet-testing system (Dr. Schleuniger Pharmatron, Switzerland). The data reported for each of the physical properties is the mean of ten individual determinations.

Disintegration times of ODTs were determined using a Hanson QC-21 disintegration test system. Deionized water (about 10 mL) was used as a disintegration media. The test was conducted at 37±0.5° C. Six ODTs per batch were analyzed and the mean is reported. This test is termed the excess water disintegration test.

Friability of ODTs having an API was measured on a VanKel Friabilator rotated at 25 rpm for 5 minutes. Twenty ODTs per batch were randomly selected for the study. The friability for each batch was calculated using following equation:

Friability (%)=(W_{b}−Wₐ)/W_{b}×100

where W_{b} and Wₐ are the weights before and after friability test.

### Dissolution Property of ODTs

Dissolution properties of the ODTs were determined by following USP 31 guidelines for each model APIs. The percent of drug released at predetermined time intervals were calculated and plotted against the sampling time to obtain the release profiles. ODTs that fall within a 70-80N hardness range were studied.

### Example 6

### ODTs with API

### ODT Formulation Design and ODT Preparation

ODTs containing model APIs were prepared at each superdisintegrant's optimum use level. Five different model APIs, which represents four different categories of drugs in terms of solubility and acidity, were chosen for this study. Four APIs were studied at relatively high dose levels and one was studied at a low dose level. Detailed API information and ODT formulations are shown in Table 6.

TABLE 6
ODT formulations that contain model API

| | | | | | |
|---|---|---|---|---|---|
| | | Dose | Group 1 | Group 2 | Group 3 |
| Ingredient | type | (mg) | (%) | (%) | (%) |

Table 6a

| | | | | |
|---|---|---|---|---|
| Pearlitol ® 200SD | Matrix | q.s. | q.s. | q.s. |
| Magnesium | Lubricant | 1.5 | 1.5 | 1.5 |
| stearate | | | | |
| Ac-Di-Sol | Disintegrant | 2.0 | | |
| crospovidone | Disintegrant | | 5.0 | |
| Glycolys | Disintegrant | | | 5.0 |

Table 6b

| | | | | | |
|---|---|---|---|---|---|
| Aspirin | API | 80 | 20 | 20 | 20 |
| Acetaminophen | API | 80 | 20 | 20 | 20 |
| Naproxen | API | 50 | 12.5 | 12.5 | 12.5 |
| Theophylline | API | 50 | 12.5 | 12.5 | 12.5 |
| Chlorpheniramine | API | 4 | 1 | 1 | 1 |
| maleate | | | | | |
| Total (%) | | | 100 | 100 | 100 |

In Table 6, the disintegrants and the APIs are presented in the alternative. That is, each formulation had at least about 73% Pearlitol® 200SD. Thus, the non-API ingredients are shown in Table 6a. The ODTs are prepared with one of the APIs from Table 6b.

Each formulation was prepared by first weighing out Pearlitol® 200 SD, disintegrant, and the model API and then premixing the ingredients in an 8 qt. Patterson Kelley V-blender for 15 minutes. Magnesium stearate was then added and followed up with additional 2 minutes mixing.

For each model API formulation, five batches of ODTs were prepared by a series of compaction forces, 4 kN, 6 kN, 8 kN, 10 kN, 12 kN, on a Stokes 512 Tablet Press with four stations. Use of the different compaction forces produced tables of different hardnesses. Standard 7/16″ concave punches and corresponding dies were used. The ODT weights were about 400 mg. SMI Director™ data acquisition system was used to record compaction process.

ODTs containing a model API were prepared to evaluate ODT characteristics for administration of an agent. Four ODTs were prepared having relatively high API dose levels (˜12%-20% by weight). Four different types of model APIs were prepared to evaluate the effect of APIs having different physical and chemical properties. In particular, aspirin was used to model highly water-soluble acidic ingredients, acetaminophen was used to model highly water-soluble basic ingredients, naproxen was used to model poorly water-soluble acidic ingredients, and theophylline was used to model poorly water-soluble basic active ingredients. For each model API formulation, a series of ODTs that cover a wide hardness range were prepared through different compaction forces (from 4 kN to 12 kN). All ODTs were tested and compared in terms of their disintegration time, hardness, friability, and API dissolution.

ODTs with API Compounds

The five model API compounds were obtained as follows: aspirin (Rhodia Group, France), acetaminophen (Rhone Poulenc, France), theophylline (Spectrum Chemicals & Laboratory Products), naproxen (Spectrum Chemicals & Laboratory Products), and chlorpheniramine maleate (Spectrum Chemicals & Laboratory Products).

### Aspirin Model ODTs

Aspirin is a highly water soluble, acidic type of active ingredient with a pKa of 3.5. The chemical structure of aspirin is shown below.

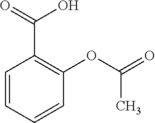

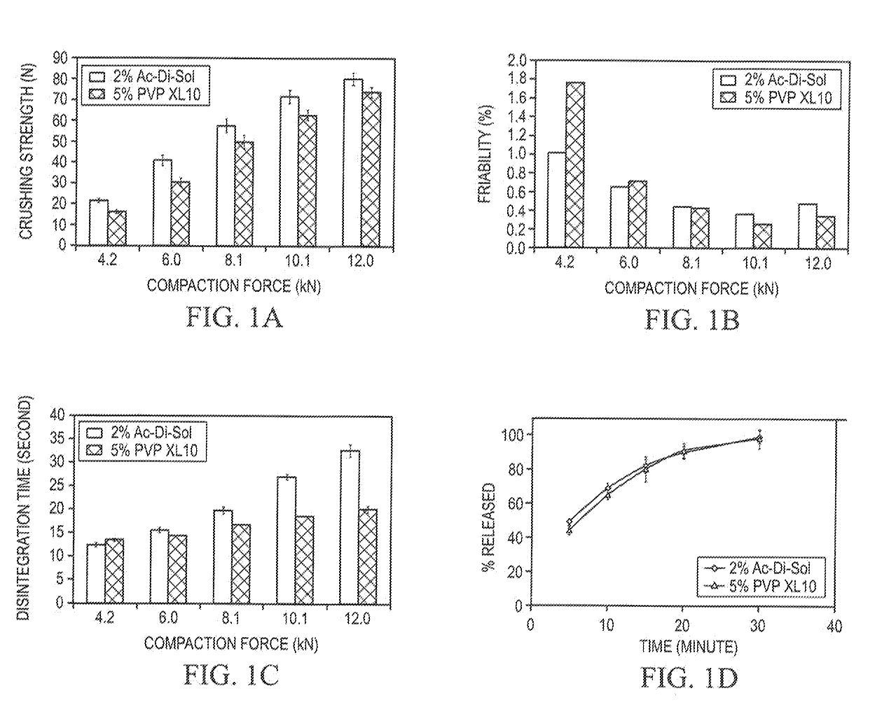

The four graphs in FIG. 1 show aspirin ODTs' crushing strength (1A), friability (1B), disintegration (1C), and dissolution (1D) results, respectively. The x-axes in graphs 1A, 1B, and 1C represents compaction forces, and the y-axes in graphs 1A, 1B, and 1C are hardness, ODT friability, and ODT disintegration time, respectively. The x-axis in Graph D is time and the y-axis is percentage of aspirin released during the time. The dark gray bars and symbols represent ODTs having 2% croscarmellose and the medium gray bars and symbols are for ODTs having 5% crospovidone (specifically PVP-XL-10). Error bars show standard deviations of the measurements.

As indicated in graph 1A, aspirin ODTs that contains 2% croscarmellose had slightly higher ODT hardness than ODTs that contains 5% PVP XL-10, over the entire compaction force range. As shown in graph 1B, in order to obtain ODTs with low friability that meet the USP requirement, a minimum of 6 kN compaction force was required, particular for 5% PVP XL-10 containing ODTs. Otherwise, special packaging would be required. Croscarmellose and crospovidone had the same disintegration rate for ODTs made at 8 kN and 10 kN compaction forces (graph 1C). No difference was found in terms of aspirin dissolution profile between ODTs that contain 2% croscarmellose and ODTs that contain 5% PVP XL-10 (graph 1D).

### Acetaminophen Model ODTs

Acetaminophen is a highly water soluble and basic active with a pKa of 9.2. Acetaminophen's chemical structure is shown below.

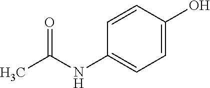

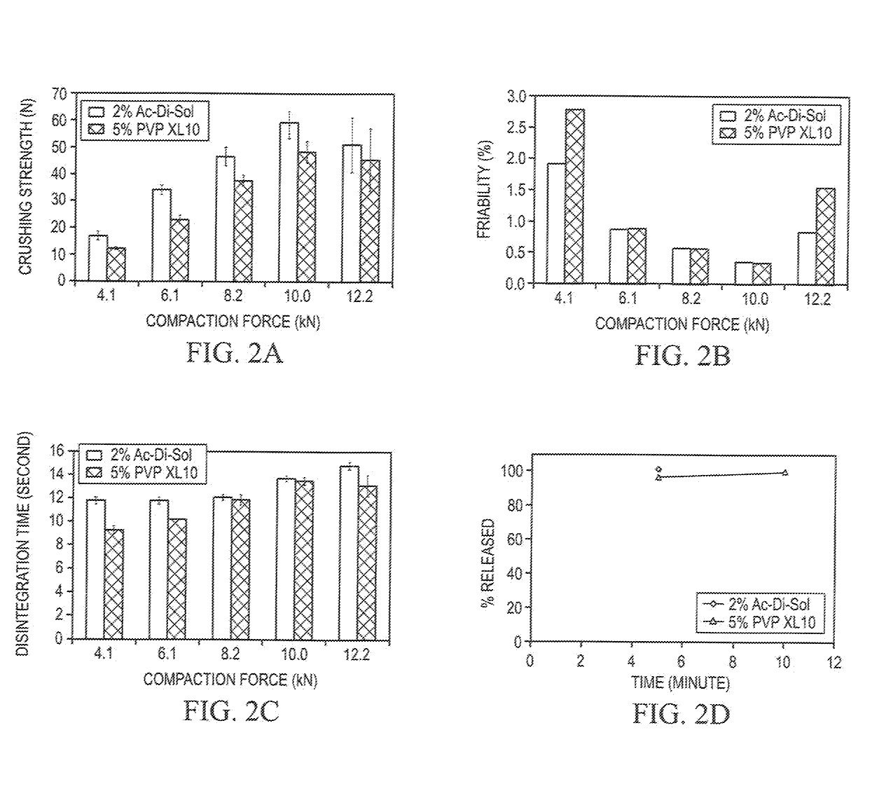

The four graphs in FIG. 2 show model acetaminophen ODTs' crushing strength (2A), friability (2B), disintegration (2C), and dissolution (2D) results, respectively. As shown graph 2A, acetaminophen ODTs were generally softer than aspirin ODTs. However, acetaminophen ODTs that contain 2% croscarmellose consistently showed higher hardness than ODTs that contain 5% PVP XL-10. As with aspirin ODTs, a minimum of 6 kN compaction force was required to produce ODTs with low friability that meet the USP requirement, especially for PVP XL-10 containing ODTs (2B). As graph 2C indicated, both 2% croscarmellose and 5% PVP XL-10 disintegrated acetaminophen ODT very rapidly, less than 20 seconds. Acetaminophen was fully released within 10 minutes and met the USP requirement. No dissolution difference was found between ODTs that contain 2% croscarmellose and ODTs that contain 5% PVP XL-10 (graph 2D).

### Naproxen Model ODTs

Naproxen is a poorly water-soluble and relative acidic API with a pKa of 4.2 and a chemical structure as shown below.

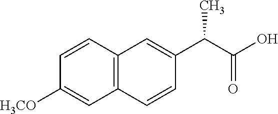

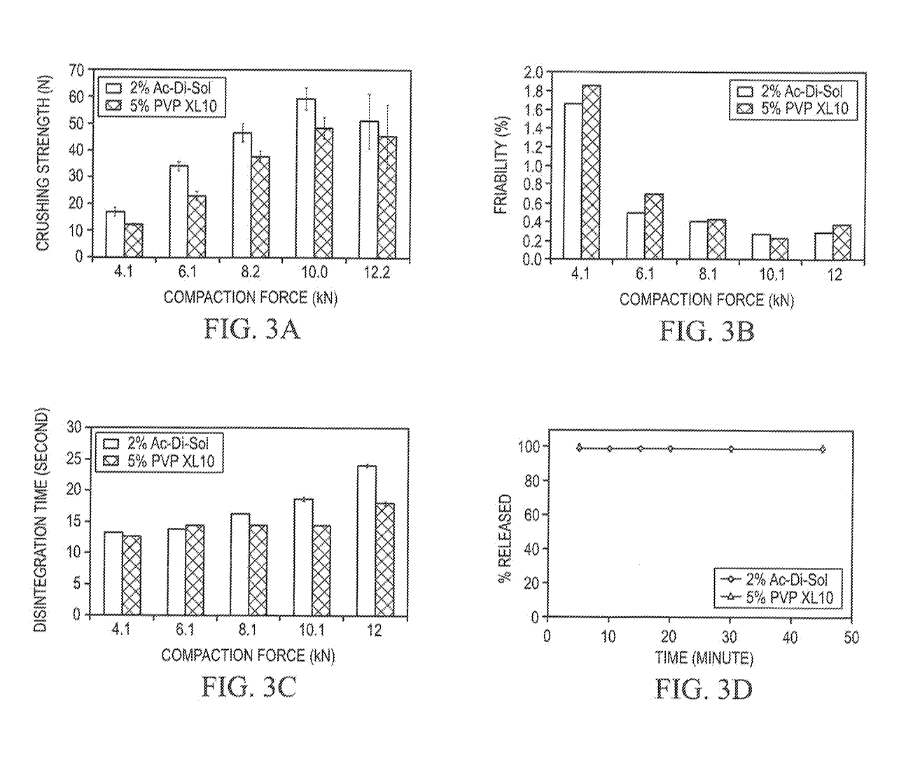

The four graphs in FIG. 3 show model naproxen ODTs' crushing strength (3A), friability (3B), disintegration (3C), and dissolution (3D) results. Overall, naproxen ODTs that contain 2% croscarmellose had slightly higher ODT hardness and lower ODT friability than ODTs that contain 5% PVP XL-10 (graph 3A and 3B). Again, a minimum of 6 kN compaction force was needed to produce ODTs with low enough friability that would not require special packaging. Both 2% croscarmellose and 5% PVP XL-10 disintegrated the naproxen ODT rapidly and met USP's 30 second requirement, particularly at common ODT hardness range (3C). Naproxen was fully released within 10 minutes and no dissolution difference was found between ODTs that contain 2% croscarmellose and ODTs that contain 5% PVP XL-10 (3D).

### Theophylline Model ODTs

Theophylline is a poorly soluble and basic drug with a pKa of 8.7. The chemical structure of theophylline is:

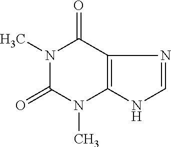

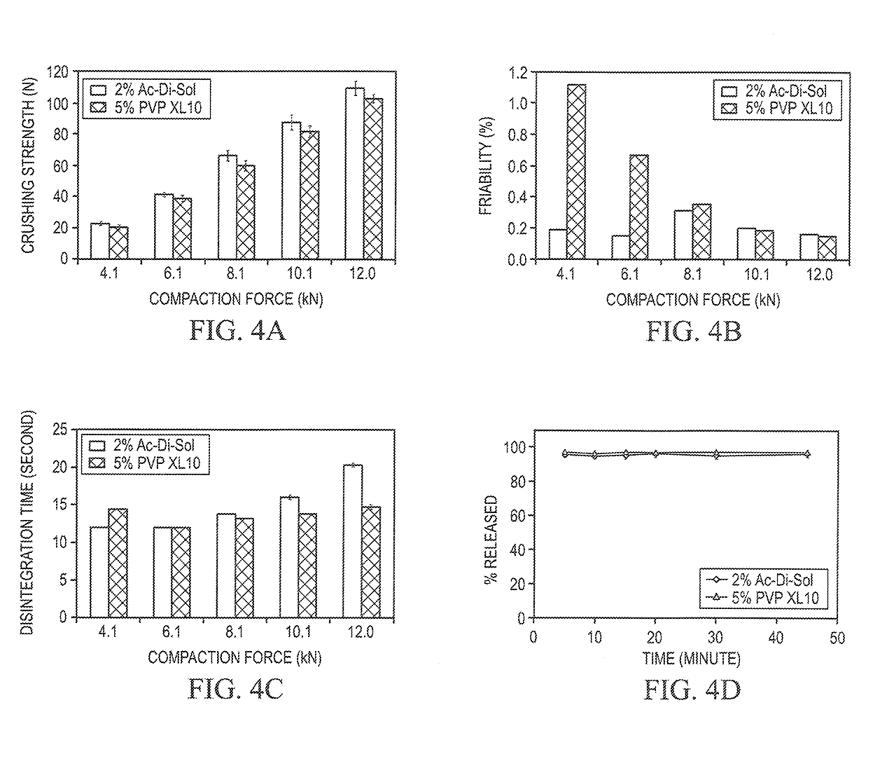

The four graphs in FIG. 4 show theophylline ODTs' crushing strength (4A), friability (4B), disintegration (4C), and dissolution (4D) results. Overall, as shown in graph 4A, theophylline ODTs that contain 2% croscarmellose had slightly higher ODT hardness than theophylline ODTs that contain 5% PVP XL-10. ODTs made with 2% croscarmellose had acceptable friability when made at all compaction forces tested. A minimum of 6 kN compaction force was required to produce ODTs with acceptable low friability, for PVP XL-10 containing ODTs. Both 2% croscarmellose and 5% PVP XL-10 disintegrated theophylline ODTs less than 30 second, over the entire compaction force range. Theophylline was fully released at five minutes and met the USP requirement. No dissolution difference was found between ODTs that contain 2% croscarmellose and ODTs that contain 5% PVP XL-10.

### Chlorpheniramine Maleate (CPM) Model ODTs

CPM is a highly soluble basic type of API with a pKa of 9.2. The chemical structure is shown below.

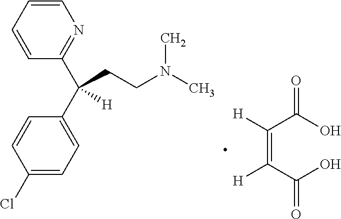

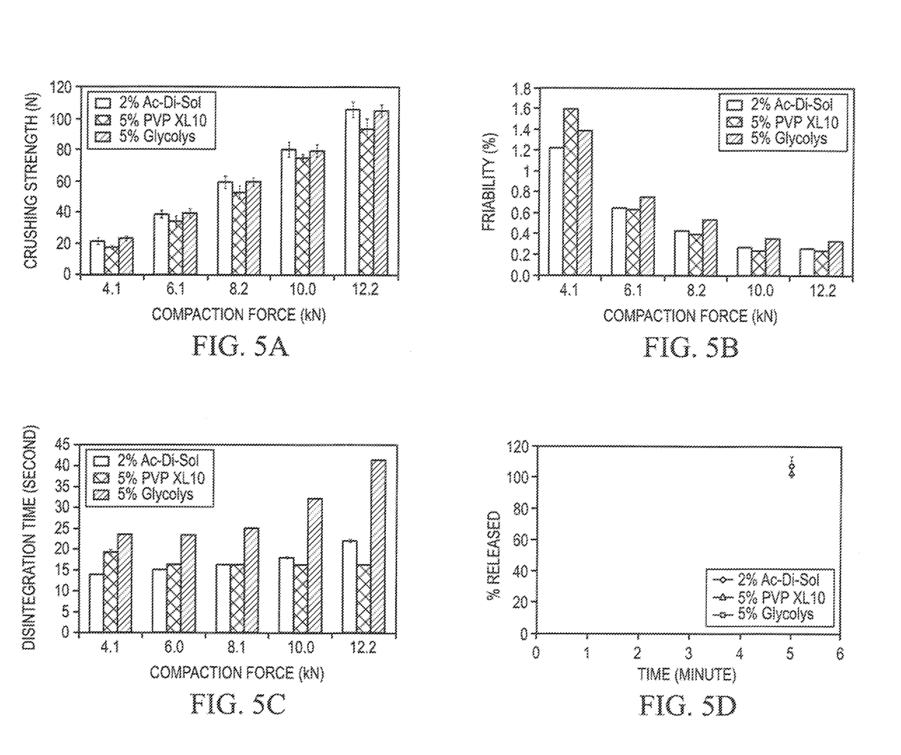

For CPM, all three types of superdisintegrants were evaluated and results are shown in FIG. 5. The four graphs in FIG. 5 show CPM ODTs' crushing strength (5A), friability (5B), disintegration (5C), and dissolution (5D) results, respectively. In general, CPM ODTs that contain 5% PVP XL-10 were slightly softer than ODTs that contain either 2% croscarmellose or 5% SSG (the latter shown in light gray bars and symbols). A minimum of 6 kN compaction force was needed to produce ODTs with acceptably low friability. 2% croscarmellose and 5% PVP XL-10 disintegrated CPM ODTs equally rapidly and both were faster than 5% SSG. CPM was fully released within 5 minutes. No dissolution difference was found among ODTs that contain different disintegrants.

Thus, although the above five API ODT formulations are very different in terms of drug solubility, acidity, and dose levels, the general findings from each formulation were similar. For example, ODTs that contain 2% croscarmellose generally showed slightly higher crushing strength and lower friability than ODTs that contain 5% PVP XL-10, which might indicate PVP XL-10 interfered more with mannitol's bonding capability than croscarmellose during compaction process. At common ODT hardness range (ODTs compressed <8 kN), all three disintegrants can achieve fast ODT disintegration that meet ODT definition at each disintegrant's optimal use level. However, 2% croscarmellose and 5% PVP XL-10 did provide faster ODT disintegration than 5% SSG. No dissolution difference was found among different superdisintegrants; they all can effectively release drug within the USP required time frame.

### Example 7

### Effect of Aging on ODT Hardness and Disintegration Time

ODTs made as in Example 2 were sealed at ambient initial relative humidity (<70), kept in sealed double plastic bags for four months or two weeks at room temperature, and then tested for dry ODT hardness and disintegration time. The ODTs showed no appreciable change in ODT weight, thickness, or diameter, which were about 400 mg, about 5 mm, and about 11 mm, respectively. The changes, after 4 months, in hardness and disintegration time of ODTs made with 1% superdisintegrant, compared to the values for ODTs at 24 hrs, are shown in Table 7.

TABLE 7
ODT Properties after 4 month storage stability - 1.0% disintegrant

| | | | Change in | Disintegration | Change in |
|---|---|---|---|---|---|
| Disintegrant | Force | Hardness | Hardness | time | Disintegration |
| (1%) | (kN) | (N) | (%) | (sec) | time (%) |
| Ac-Di-Sol ® | 4.14 | 52.0 | 109.7 | 13.2 | 15.8 |
| Ac-Di-Sol ® | 6.15 | 88.2 | 114.1 | 16.5 | 22.2 |
| Ac-Di-Sol ® | 8.11 | 121.1 | 98.5 | 25.7 | 42.8 |
| Ac-Di-Sol ® | 10.15 | 145.2 | 89.8 | 31.3 | 19.0 |
| Ac-Di-Sol ® | 12.11 | 167.6 | 75.0 | 47.5 | 50.8 |
| Glycolys ® | 3.98 | 40.0 | 47.6 | 19.6 | 14.6 |
| Glycolys ® | 5.96 | 69.1 | 48.6 | 26.4 | 17.9 |
| Glycolys ® | 8.09 | 104.6 | 43.7 | 31.5 | 4.0 |
| Glycolys ® | 10.09 | 136.8 | 44.0 | 43.8 | 8.2 |
| Glycolys ® | 12.11 | 158.5 | 30.6 | 60.2 | 17.8 |
| Polyplasdone ® | 4.03 | 37.5 | 61.6 | 110.3 | −1.0 |
| XL | | | | | |
| Polyplasdone ® | 6.09 | 67.2 | 52.4 | 202.2 | 5.5 |
| XL | | | | | |
| Polyplasdone ® | 8.06 | 99.3 | 58.9 | 221.5 | −10.8 |
| XL | | | | | |
| Polyplasdone ® | 10.14 | 130.9 | 62.4 | 221.3 | 1.8 |
| XL | | | | | |
| Polyplasdone ® | 12.11 | 152.5 | 53.3 | 211.0 | −2.4 |
| XL | | | | | |
| Kollidon ® CL- | 4.11 | 64.5 | 124.7 | 241.1 | −0.6 |
| SF | | | | | |
| Kollidon ® CL- | 6.02 | 98.6 | 119.1 | 297.3 | 6.5 |
| SF | | | | | |
| Kollidon ® CL- | 8.08 | 134.5 | 102.3 | 273.3 | 20.5 |
| SF | | | | | |
| Kollidon ® CL- | 10.11 | 161.4 | 83.2 | 264.5 | 13.8 |
| SF | | | | | |
| Kollidon ® CL- | 12.14 | 186.8 | 84.4 | 210.2 | 13.6 |
| SF | | | | | |

The changes, after 4 months, in hardness and disintegration time of ODTs made with 2% superdisintegrant are shown in Table 8.

TABLE 8
ODT Properties after 4 month storage stability - 2.0% disintegrant

| | | | Change in | Disintegration | Change in |
|---|---|---|---|---|---|
| Disintegrant | Force | Hardness | Hardness | time | Disintegration |
| (2%) | (kN) | (N) | (%) | (sec) | time (%) |
| Ac-Di-Sol ® | 3.93 | 36.3 | 44.6 | 13.2 | 12.8 |
| Ac-Di-Sol ® | 6.10 | 68.8 | 43.0 | 13.8 | 1.5 |
| Ac-Di-Sol ® | 8.18 | 103.3 | 40.4 | 17.1 | 14.0 |
| Ac-Di-Sol ® | 10.19 | 132.4 | 37.1 | 21.6 | 19.3 |
| Ac-Di-Sol ® | 12.20 | 162.9 | 42.0 | 27.3 | 19.2 |
| Glycolys ® | 4.01 | 37.4 | 47.2 | 18.6 | 16.3 |
| Glycolys ® | 6.05 | 68.8 | 45.5 | 21.6 | 6.9 |
| Glycolys ® | 8.09 | 100.9 | 42.7 | 29.3 | 14.5 |
| Glycolys ® | 10.05 | 132.9 | 44.6 | 36.9 | 13.5 |
| Glycolys ® | 12.15 | 161.1 | 36.8 | 50.4 | 26.3 |
| Polyplasdone ® | 4.01 | 32.8 | 53.3 | 13.80 | 6.2 |
| XL | | | | | |
| Polyplasdone ® | 6.05 | 60.1 | 52.2 | 18.00 | −10.9 |
| XL | | | | | |
| Polyplasdone ® | 8.04 | 94.0 | 55.4 | 31.00 | 25.5 |
| XL | | | | | |
| Polyplasdone ® | 10.01 | 120.8 | 71.1 | 45.80 | 26.2 |
| XL | | | | | |
| Polyplasdone ® | 12.04 | 147.9 | 46.9 | 69.20 | 13.6 |
| XL | | | | | |
| Kollidon ® CL- | 4.04 | 50.8 | 39.3 | 11.7 | 33.0 |
| SF | | | | | |
| Kollidon ® CL- | 6.11 | 88.2 | 41.2 | 15.6 | 28.9 |
| SF | | | | | |
| Kollidon ® CL- | 8.12 | 122.8 | 40.9 | 21.3 | 12.1 |
| SF | | | | | |
| Kollidon ® CL- | 10.11 | 149.0 | 33.1 | 36.3 | 35.5 |
| SF | | | | | |
| Kollidon ® CL- | 12.18 | 189.1 | 31.8 | 48.7 | 4.1 |
| SF | | | | | |

Thus, storage for four months increases the hardness of ODTs prepared with croscarmellose, although the effect is more moderate for ODTs having 2% croscarmellose. Despite the increased hardness, ODTs prepared with croscarmellose had acceptable disintegration times. In contrast, the acceptable window of compression for ODTs made with 1% of the other tested superdisintegrants was much narrower or non-existent. With regard to ODTs made with 2% superdisintegrant, croscarmellose provided the broadest range of compression forces that provided an ODT with an acceptable disintegration time.

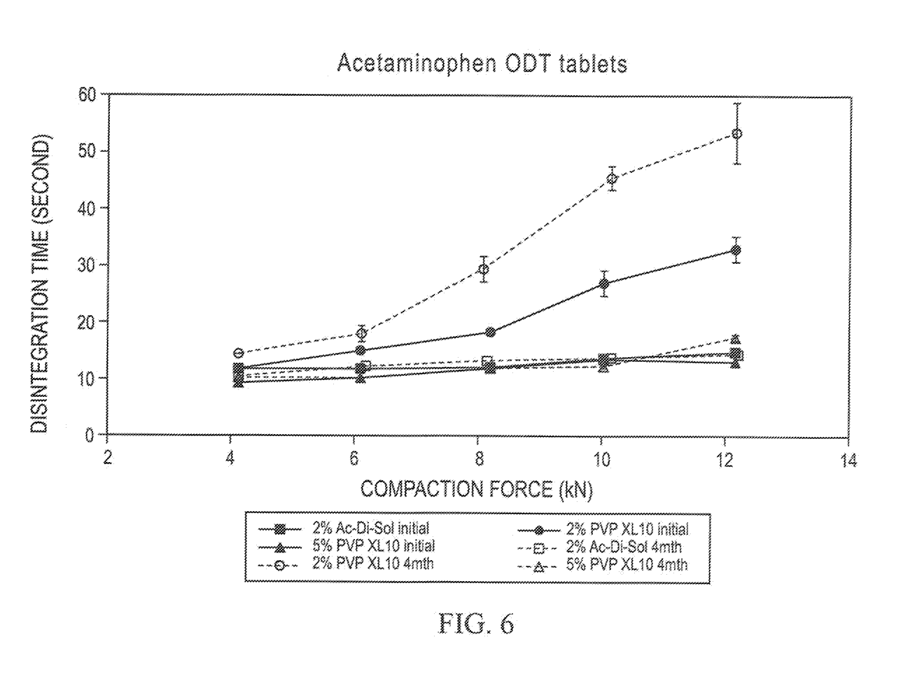

FIG. 6 shows an example of the effect on ODT disintegration of aging on ODTs prepared with croscarmellose and crospovidone. Specifically, the ODT are prepared with 20% (wt/wt) acetaminophen.

The 4 month storage data may be compared with 2 week storage data. Some 11 mm standard concave ODTs were kept in sealed containers for two weeks at room temperature. ODTs made at 4 to 12 kN compaction forces had the following changes in hardness: 0.5% Ac-Di-Sol®, 0.4 to 7.6%; 1.0% Ac-Di-Sol®, minus 8.7 to +7.2%; 1.5% Ac-Di-Sol®, 28.2 to 36.5%; and 2% Ac-Di-Sol®, 16.4 to 23.5%. Disintegration times for all the Ac-Di-Sol® ODTs prepared at 4 to 10 kN and stored for two weeks were less than 30 sec, except for ODTs having 0.5% Ac-Di-Sol® prepared at 10 kN compaction force. In contrast, all the ODTs prepared at 4 to 12 kN compaction force using 0.5% (wt/wt) of Polyplasdone® XL, Polyplasone® XL-10, Kollidon® CL, Kollidon® CL-F, or Kollidon® CL-SF had disintegration times in excess of 30 sec after storage for two weeks.

Tables 9 and 10 show the effect of storage for two weeks on the hardness and disintegration time of standard concave 11 mm ODTs made with mannitol, magnesium stearate, and selected disintegrants.

TABLE 9
ODT Properties after 2 week storage - 1.0% disintegrant

| | | | | | Change in |
|---|---|---|---|---|---|
| | Compaction | | Change in | Disintegration | Disintegration |
| Disintegrant | Force | Hardness | Hardness | Time | Time |
| (1% wt/wt) | (kN) | (N) | (%) | (second) | (%) |
| Ac-Di-Sol | 4.1 | 24.50 | −1.21 | 11.7 | 2.63 |
| Ac-Di-Sol | 6.1 | 42.60 | 3.40 | 14.6 | 8.15 |
| Ac-Di-Sol | 8.1 | 55.70 | −8.69 | 20.9 | 16.11 |
| Ac-Di-Sol | 10.1 | 82.00 | 7.19 | 25.8 | −1.90 |
| Ac-Di-Sol | 12.1 | 94.80 | −1.04 | 34 | 7.94 |
| Glycolys | 4.0 | 19.0 | −29.89 | 16.2 | −5.26 |
| Glycolys | 6.0 | 34.0 | −26.88 | 21.3 | −4.91 |
| Glycolys | 8.1 | 52.6 | −27.75 | 31.1 | 2.64 |
| Glycolys | 10.1 | 69.6 | −26.74 | 38.8 | −4.20 |
| Glycolys | 12.1 | 89.2 | −26.52 | 52 | 1.76 |
| Polyplasone ® | 4.0 | 30.90 | 33.19 | 97.8 | −12.21 |
| XL-10 | | | | | |
| Polyplasone ® | 6.1 | 56.00 | 26.98 | 220.8 | 15.18 |
| XL-10 | | | | | |
| Polyplasone ® | 8.1 | 80.50 | 28.80 | 244.7 | −1.41 |
| XL-10 | | | | | |
| Polyplasone ® | 10.1 | 101.20 | 25.56 | 208 | −4.37 |
| XL-10 | | | | | |
| Polyplasone ® | 12.1 | 129.60 | 30.25 | 210.5 | −2.59 |
| XL-10 | | | | | |
| Kollidon ® CL- | 4.0 | 28.5 | 7.55 | 151.7 | −9.92 |
| SF | | | | | |
| Kollidon ® CL- | 6.1 | 47.8 | 4.09 | 237.9 | 23.71 |
| SF | | | | | |
| Kollidon ® CL- | 8.1 | 69.6 | 2.35 | 228.4 | 3.30 |
| SF | | | | | |
| Kollidon ® CL- | 10.1 | 91.7 | 3.85 | 199.5 | 7.95 |
| SF | | | | | |
| Kollidon ® CL- | 12.2 | 119.1 | 6.65 | 194.4 | 6.99 |
| SF | | | | | |

TABLE 10
ODT Properties after 2 week storage - 2.0% disintegrant

| | | | | | Change in |
|---|---|---|---|---|---|
| | Compaction | | Change in | Disintegration | Disintegration |
| Disintegrant | Force | Hardness | Hardness | Time | Time |
| (2% wt/wt) | (kN) | (N) | (%) | (second) | (%) |
| Ac-Di-Sol | 3.9 | 31.0 | 23.51 | 12.3 | 5.13 |
| Ac-Di-Sol | 6.1 | 56.9 | 18.30 | 14.4 | 5.88 |
| Ac-Di-Sol | 8.2 | 87.1 | 18.34 | 16.2 | 8.00 |
| Ac-Di-Sol | 10.2 | 112.4 | 16.40 | 20.1 | 11.05 |
| Ac-Di-Sol | 12.2 | 134.1 | 16.91 | 24.6 | 7.42 |
| Glycolys | 4.0 | 18.3 | −27.95 | 16.2 | 1.25 |
| Glycolys | 6.1 | 35.1 | −25.79 | 20.4 | 0.99 |
| Glycolys | 8.1 | 54.0 | −23.62 | 27.1 | 5.86 |
| Glycolys | 10.1 | 72.5 | −21.11 | 33.3 | 2.46 |
| Glycolys | 12.1 | 85.0 | −27.84 | 43.9 | 10.03 |
| Polyplasdone ® | 4.0 | 26.5 | 23.83 | 14.7 | 13.08 |
| XL-10 | | | | | |
| Polyplasdone ® | 6.1 | 50.2 | 27.09 | 16.6 | −17.82 |
| XL-10 | | | | | |
| Polyplasdone ® | 8.0 | 76.9 | 27.11 | 27.1 | 9.72 |
| XL-10 | | | | | |
| Polyplasdone ® | 10.0 | 96.2 | 36.26 | 43 | 18.46 |
| XL-10 | | | | | |
| Polyplasdone ® | 12.0 | 118.8 | 17.95 | 71.8 | 17.90 |
| XL-10 | | | | | |
| Kollidon ® CL- | 4.0 | 28.0 | 14.75 | 10.8 | 22.73 |
| SF | | | | | |
| Kollidon ® CL- | 6.1 | 45.6 | 0.88 | 12.2 | 0.83 |
| SF | | | | | |
| Kollidon ® CL- | 8.2 | 73.0 | 7.51 | 18.3 | −3.68 |
| SF | | | | | |
| Kollidon ® CL- | 10.2 | 95.7 | 4.48 | 30 | 11.94 |
| SF | | | | | |
| Kollidon ® CL- | 12.2 | 127.4 | 12.15 | 68.3 | 45.94 |
| SF | | | | | |

### Example 8

### ODT Disintegration in a Small Volume

A method was developed to evaluate disintegration of an ODT in a small volume of fluid, and thereby simulate disintegration in the mouth.

### Texture Analyzer Evaluation of ODTs

The USP disintegration test method is well established to differentiate traditional tablet's disintegration time, but it does not really reflect the disintegration environment of an ODT in normal usage, in which there is typically a small amount of fluid available. In order to mimic the ODTs disintegrating or softening process in the oral cavity, a method was developed using a texture analyzer to monitor the disintegrating process of ODTs in 2 mL of deionized water.

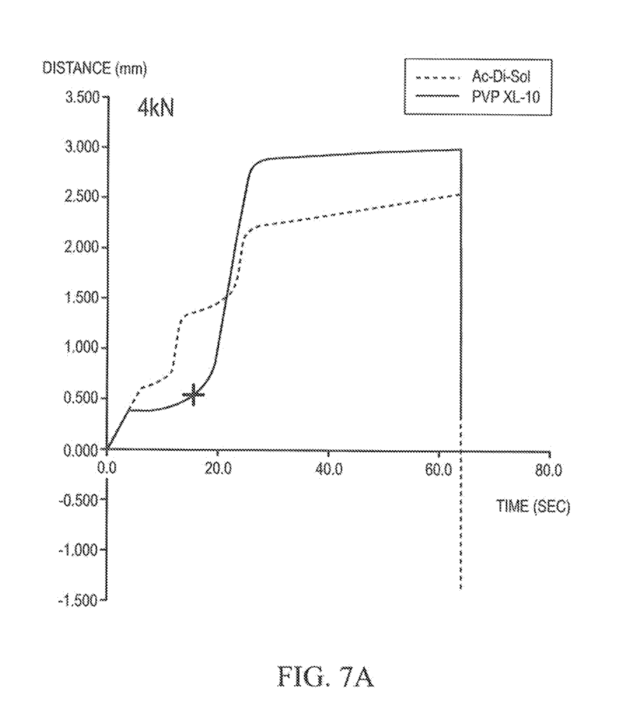

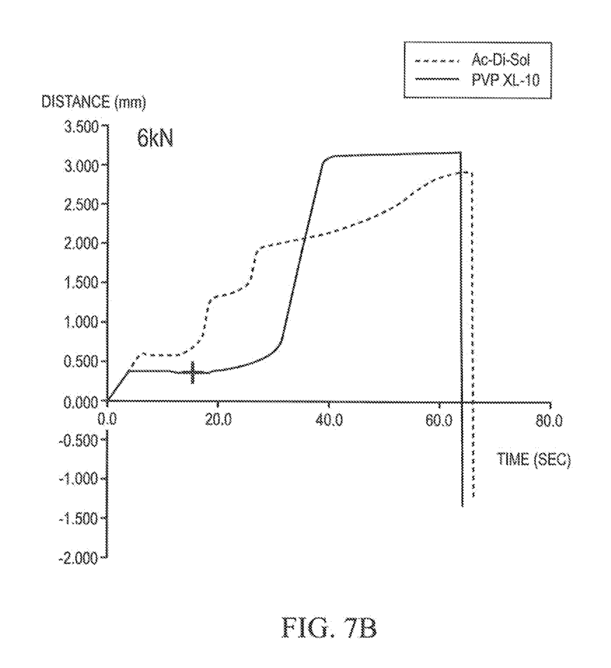

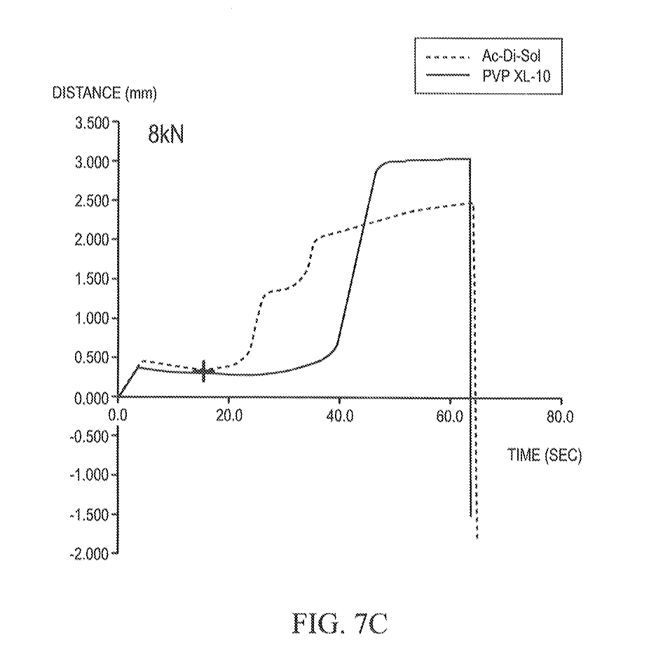

FIG. 7 compares a small fluid volume ODT disintegration process in commonly used ODT hardness ranges between ODTs that contain 2% croscarmellose and ODTs that contains 5% PVP XL-10. The three panels show ODTs consisting of superdisintegrant, mannitol, and magnesium stearate that were compressed at a) 4 kN, b) 6 kN, and c) 8 kN, respectively. Each panel shows the probe travel distance as a function of ODT time in contact with shallow water. The plateau portion of each curve indicates the ODT was cohesive enough to withstand the very slight compressive force from the probe before it further disintegrated. The steep vertical portion(s) of each curve indicate(s) that the ODT had disintegrated further and the probe distance increased in search of the target resistance force. The test came to an end when the probe touched the bottom of the weighing boat. For ODTs of 4 kN, 6 kN, and 8 kN, as illustrated, the ODTs prepared with croscarmellose had initial disintegration earlier than the PCP XL-10 ODTs. For 4 kN ODTs, the croscarmellose ODTs showed initial disintegration at about 15 sec whereas the PVP XL-10 ODTs did not show initial disintegration until after about 20 sec. The croscarmellose ODTs made at 6 kN showed initial disintegration at about 18 sec, whereas the PVP XL-10 ODTs did not show initial disintegration until about 30 sec. The croscarmellose ODTs made at 8 kN showed initial disintegration at about 22 sec, whereas the PVP XL-10 ODTs did not show initial disintegration until about 40 sec. Moreover, disintegration of croscarmellose ODTs was substantially completed before the PVP XL-10 disintegration.

The small volume disintegration assay using a texture analyzer showed that 2% croscarmellose and 5% PVP XL-10 provided different ODT disintegration patterns. ODTs prepared with croscarmellose had an earlier initiation of disintegration than the PCP-XL10 ODTs. Also, croscarmellose containing ODTs showed an alternative pattern in the plateau and vertical portions of the disintegration curve, indicating a gradual softening or disintegration process. On the other hand, PVP XL-10 containing ODTs displayed a longer initial plateau followed by a big vertical portion, indicating an initial delay in ODT softening or disintegration, followed by a sudden ODT softening or disintegration. This finding partially explained the mouth feel results. That is, out of all panelists who made the correct judgment, 80% commented that croscarmellose containing ODTs provided a preferred mouth feel to PVP XL-10 containing ODTs, such as a smoother mouth feel with faster disintegration, which is likely attributable to the rapid and gradual disintegration pattern of croscarmellose containing ODTs.

Thus, among the three types of commercial super-disintegrants, croscarmellose, crospovidone, and SSG, croscarmellose offers clear advantages for use at very low amounts in ODT applications. The optimal level of superdisintegrant is about 2% for croscarmellose, 5% for crospovidone, and 5% for SSG. Furthermore, croscarmellose, particularly Ac-Di-Sol®, is the most effective one among all three types of superdisintegrants in that it achieved disintegration rapidly at the lowest use level and more effectively disintegrated harder ODTs. In general, ODTs that contain 2% croscarmellose had the same mouth feel as ODTs that contain 5% PVP XL-10, however, ODTs made with 2% croscarmellose and 5% PVP XL-10 disintegrated in a different pattern when only a small amount of water was available, such as in the oral cavity.

### Example 9

### ODT Lacking Lubricant

The ODT can be prepared without lubricant. Thus, spray dried mannitol is blended with acetaminophen (to 20% wt/wt, as API), sodium croscarmellose (to 1% wt/wt, as superdisintegrant), sucralose (0.2% wt/wt), and black cherry flavor (0.5% wt/wt). The final composition is compressed at 4 kN, 6 kN, or 8 kN compaction force to form 3/8 “ ODTs of acceptable properties.

### Example 10

### ODTs of 300 mg

ODTs were prepared as ⅜″ standard concave tablets of 300 mg each. The ODTs were made at 3 kN, 5 kN, or 7 kN compaction forces and had 2% (wt/wt) croscarmellose, 1.5% (wt/wt) magnesium stearate, and the balance mannitol in one of the following flavors: black cherry, strawberry, raspberry, or pineapple (0.5% wt/wt). All these tablets had disintegration times (in excess water) of less than 30 sec and tensile strengths between 0.4 MPa and 1.1 MPa. All friabilities were less than 0.6%.

### Example 11

### ODTs of 200 mg

ODTs were prepared as ⅜″ standard concave tables of 200 mg each. The ODTs were made with 2% (wt/wt) croscarmellose, 1.5% (wt/wt) magnesium stearate, and the balance mannitol, having either black cherry or pineapple flavor. The ODTs were prepared at compaction forces from 2.5 kN to 5 kN. All these ODTs disintegrated in less than 30 sec in the excess water volume test. The tensile strengths ranged from 0.9 MPa to 1.6 MPa. Friability values were between 0.2 and 0.36%.

### Example 12

### ODTs of 100 mg

ODTs were prepared as ¼″ standard concave tablets of 100 mg each. The ODTs were made using a compaction force of 2.5 kN with a composition of 2% croscarmellose, 1.5% (wt/wt) magnesium stearate, and the balance mannitol in either of two flavors: black cherry or pineapple. The disintegration times were less than 30 sec using the excess water test. The tensile strengths for these ODTs were from 1.1 to 1.3 MPa. Friability values were 0.18% to 0.19%.

Having now fully described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims. All publications, patents, and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

It is claimed:

1. An aged direct compression orally disintegrating tablet (ODT) comprising:
a) between about 0.3% to about 2% (wt/wt) sodium croscarmellose relative to the total weight of the ODT,
b) a polyol,
c) optionally a lubricant, and
d) an active pharmaceutical or nutraceutical ingredient (API),
wherein after storage for four months a tensile strength of the ODT is at least 0.5 MPa and a disintegration time using an excess water test is less than 30 seconds.

2. The ODT of claim 1, wherein the polyol is spray dried mannitol.

3. The ODT of claim 2 further comprising one or more colorants, sweeteners, fragrances, flavor compounds, and/or flavor blockers.

4. The ODT of claim 2 wherein the API comprises up to about 20% (wt/wt) of the ODT.

5. The ODT of claim 4, wherein the spray dried mannitol comprises the balance of the ODT.

6. The ODT of claim 4, wherein the API is selected from the group consisting of an acidic poorly water soluble API, a basic poorly water soluble API, an acidic water soluble API, and a basic water soluble API, or a combination thereof.

7. The ODT of claim 4, wherein the API is an acidic poorly water soluble API.

8. The ODT of claim 4, wherein the API is a basic poorly water soluble API.

9. The ODT of claim 4, wherein the API is an acidic water soluble API.

10. The ODT of claim 4, wherein the API is a basic water soluble API.

11. The ODT of claim 4, wherein the disintegration time is less than 20 seconds.

12. The ODT of claim 4, wherein the tensile strength is from 0.85 to 1.7 MPa.

13. The ODT of claim 4, wherein the tensile strength is from 1.0 to 1.5 MPa.

14. The ODT of claim 1, wherein the disintegration time is less than 20 seconds.

15. The ODT of claim 1, wherein the lubricant is magnesium stearate.

16. The ODT of claim 1, further comprising 0.1% to 8% (wt/wt) microcrystalline cellulose.

17. A direct compression ODT, consisting essentially of about 0.5% (wt/wt) to 2.0% (wt/wt) sodium croscarmellose relative to the total weight of the ODT, from 0.1% (wt/wt) to 2.0% (wt/wt) magnesium stearate, up to about 20% (wt/wt) of an API, optionally from 0.1% to less than 10% (wt/wt) microcrystalline cellulose, optionally one or more colorants, sweeteners, fragrances, flavor compounds, and/or flavor blockers, and the balance spray-dried mannitol,
a) wherein the dry ODT has a tensile strength from 0.5 to 1.7 MPa,
b) wherein the disintegration time using an excess water test is less than 30 seconds, and
c) wherein the friability is less than 0.5%.

18. The ODT of claim 17, wherein the disintegration time is less than 20 seconds.

19. The ODT of claim 17, wherein the tensile strength is greater than 1.0 MPa.

20. The ODT of claim 17, wherein the ODT weight is greater than 60 mg.

21. A direct compression ODT comprising from about 0.5% (wt/wt) to 2.0% (wt/wt) superdisintegrant relative to the total weight of the ODT, a lubricant, optionally an API, optionally one or more colorants, sweeteners, fragrances, flavor compounds, and/or flavor blockers, and the balance spray-dried polyol,
a) wherein the dry ODT has a tensile strength from 1.0 to 1.5 MPa,
b) wherein the friability is less than 0.5%, and
c) wherein the ODT disintegration using a 2 mL water test begins at less than 20 seconds.

22. The ODT of claim 21, wherein the superdisintegrant is sodium croscarmellose.

23. The ODT of claim 21, further comprising that the ODT lacks any disintegrant other than the superdisintegrant.

24. The ODT of claim 21, wherein the API comprises less than about 20% (wt/wt) of the ODT.
